(19) United States
(12) Patent Application Publication
(10) Pub. No.: US 2007/0225337 A1
(43) Pub. Date: Sep. 27, 2007

(54) Novel Co-Precipitate of Amorphous Rosiglitazone

(75) Inventors: Julia Greil, Kramsach (AT); Siegfried Wolf, Brixlegg (AT); Johannes Ludescher, Breitenbach (AT)

Correspondence Address: NOVARTIS;CORPORATE INTELLECTUAL PROPERTY, ONE HEALTH PLAZA 104/3, EAST HANOVER, NJ, 07936-1080, US

(73) Assignee: SANDOZ AG, Basle, CH (CH)

(21) Appl. No.: 10/599,567

(22) PCT Filed: Mar. 30, 2005
(86) PCT No.: PCT/EP05/03332

§ 371 Date: Oct. 2, 2006

(30) Foreign Application Priority Data
Mar. 31, 2004 (SI) P200400101
Sep. 17, 2004 (SI) P200400258

(51) Int. Cl. A61K 31/44; C07D 417/02

(52) U.S. Cl. 514/342; 546/269.7

(57) ABSTRACT

A novel coprecipitate of amorphous rosiglitazone maleate with a pharmaceutically acceptable carrier, e.g. polyvinylpyrolidone, mannitol, lactose, methylcellulose, cyclodextrin or silicon dioxide, a process for the preparation of said novel co-precipitate and the use of said novel coprecipitate of amorphous rosiglitazone with a pharmaceutically acceptable carrier in the treatment and/or prophylaxis of diabetes mellitus, conditions associated with diabetes mellitus and certain complications thereof, are disclosed. A novel solid solution of rosiglitazone maleate with a pharmaceutically acceptable carrier, preferably with polyethylene glycol PEG from 4000 to 40.000 of average mol. wt., a process for the preparation thereof and use are disclosed. A novel coprecipitate of amorphous rosiglitazone maleate with a pharmaceutically acceptable carrier and a novel solid solution of rosiglitazone maleate with a pharmaceutically acceptable inert carrier are stable and may be particularly suitable for bulk preparation, handling and formulation advantages.

### FIELD OF THE INVENTION

(Int. Cl.: C07 D 417/12, A61 K 31/425, A61 K 31/44)

The present invention relates to a novel stable coprecipitate of amorphous form of 5-[4-[2-[N-methyl-N-(2-pyridyl)amino]ethoxy]benzyl]thiazolidine-2,4-dione maleate having formula (I), (hereinafter referred as rosiglitazone maleate) with a pharmaceutically acceptable carrier, to a pharmaceutical composition comprising said novel coprecipitates, to a process for the preparation of said novel coprecipitate and to its use in medicine. In another aspect the invention relates to a novel solid solution of rosiglitazone maleate with a pharmaceutically acceptable carrier. Both amorphous precipitation and solid solution in an inert carrier are included in the general term “solid dispersion systems”. A novel coprecipitate of amorphous rosiglitazone maleate with a pharmaceutically acceptable inert carrier and a novel solid solution of rosiglitazone maleate in an inert carrier are useful for the treatment and/or prophylaxis of diabetes mellitus, conditions associated with diabetes mellitus and certain complications thereof. Diabetes mellitus preferably means Type II diabetes mellitus.

### TECHNICAL PROBLEM

There is a constant need for preparing stable pharmaceutical dosage form comprising rosiglitazone maleate which would be particularly suitable for bulk preparation, handling and formulation advantages.

### PRIOR ART

Rosiglitazone is a well-known active compound, described in EP 306228 A1 also in a tautomeric form and/or its pharmaceutically acceptable salt thereof, and/or a pharmaceutically acceptable solvate thereof, useful for the treatment and/or prophylaxis of hyperglycaemia, hyperlipidaemia or hypertension.

PCT application, publication number WO 94/05659, discloses certain thiazolidinedione derivatives or a tautomeric form thereof and/or a pharmaceutically acceptable solvate thereof, including 5-[4-[2-(N-methyl-N-(2-pyridyl)amino]ethoxy]benzyl]thiazolidine-2,4-dione maleic acid salt (rosiglitazone maleate) as preferred compound, which is active compound in commercial drug Avandia®. Rosiglitazone maleate is significantly more soluble in water than the corresponding free base and show also good stability in the solid form.

PCT applications, publ. numbers WO 99/31093, WO 99/31094 and WO 99/31095 each disclose novel hydrates of rosiglitazone maleate, a process for the preparation of such a compound, a pharmaceutical composition containing such a compound and the use of such a composition for the treatment of diabetes mellitus.

PCT applications, publ. numbers WO 00/64892, WO 00/64893 and WO 00/64896 describe novel polymorphs of rosiglitazone maleate, a process for preparing such polymorphs, a pharmaceutical composition containing such polymorphs and the use of such polymorphs for the treatment of diabetes mellitus.

PCT application, publ. number WO 02/26737, discloses novel polymorphic/pseudopolymorphic forms I-IV of rosiglitazone maleate, a pharmaceutical composition comprising one of the novel polymorphic form or their mixture and a pharmaceutically acceptable carrier.

PCT application, publ. No. WO 04/014304 describes a pharmaceutical composition comprising an electrospun fiber of a pharmaceutically acceptable polymeric carrier homogeneously integrated with a stable amorphous form of a pharmaceutically acceptable active agent and the process of making polymer nanofibers from either a solution or melt under electrical forces, to prepare stable solid dispersions of amorphous drugs in polymer nanofibers as well. Among the numerous active agents amorphous rosiglitazone may be used.

PCT application, publ. No. WO 04/062667 describes an amorphous rosiglitazone maleate and preparation and use thereof for a pharmaceutical composition and a method for medical treatment including combination therapy.

Solid dispersions are well known in Farmacy and are described in the literature, e.g. Mahdu K. Vadnere, Coprecipitates and Melts, Encyclopedia of Pharmaceutical Technology (Editors James Swarbrick and James C. Boylan), Vol. 3, 1990, pp. 337-352, or in the article D. W. Bloch and P. P. Speiser, Pharm. Acta. Helv. 62, No. 1, (1987), pp 23-27.

The term “solid dispersion” refers to “the dispersion of one or more active ingredients in an inert carrier or matrix at solid state, prepared by the melting, solvent, or melting-solvent method”. The term “solid dispersion” includes six systems, including “amorphous precipitations in a carrier and solid solutions”.

Poorly soluble drugs have often low absorption and week bioavailability. Several possibilities of increasing solubility and improve dissolution rate of poorly soluble drugs are known, including the preparation of amorphous form thereof.

Coprecipitates and melts (solid solution) are solid dispersions that result to reduced particles size to the molecular level. Above literature teach that by preparing amorphous precipitations in a crystalline carrier the drug is precipitated out in an amorphous form in the former as opposed to a crystalline form of the latter.

Two procedures used to prepare solid dispersions are melting or fusion method and solvent method. The first method describes that a physical mixture of an active agent and water-soluble carrier is heated until it is melt. The melt is solidified rapidly under cooling and rigorous stirring and subsequently isolation of desired solid solution. The melting method requires both the drug and carrier to be thermally stable at the processing temperature. The advantage of said method is that no solvents are used.

Solvent method is used to prepare solid dispersions of active agent in suitable polymer by using solvents. The solvent is usually removed by evaporation under reduced pressure at varying temperatures, but other methods for removal the solvents may be used as well, e.g. spray drying. The major advantage of the solvent method is that thermal decomposition of drugs and inert carriers associated with the melting method can be avoided.

Solid dispersions of poorly soluble drug prepared by above described methods usually exhibit higher dissolution rates than the starting crystalline drug but may be hindered by dissolution in case of using high molecular weight polymers as carriers. Several solid dispersion systems are on the market, e.g. solid dispersion of nifedipine with PVP (Nifelan®).

### DESCRIPTION OF THE INVENTION

The object of the present invention is to find a novel stable pharmaceutical form of rosiglitazone maleate, which would be particularly suitable for bulk preparation, handling and formulation advantages. We have surprisingly and unexpectedly found that this problem was solved by a novel stable coprecipitate of amorphous rosiglitazone maleate with a pharmaceutically acceptable carrier.

The higher aqueous solubility of the amorphous form results to a higher rate of dissolution, and to better oral bioavailability. Because of instability of amorphous form this may be overcome by preparing said novel coprecipitate in order to stabilize the amorphous form of rosiglitazone maleate.

As pharmaceutically acceptable carriers for preparing an amorphous coprecipitate of the invention may be used any materials described in above cited Encyclopedia of Pharmaceutical Technology (Vol. 3, Table 1 on page 345), but preferably carriers selected from the group consisting of polyvinylpyrrolidone (PVP), silicon dioxide, mannitol, lactose, methylcellulose and a cyclodextrine. The cyclodextrine may be any member of broad group of natural α, β and gamma cyclodextrines or semisynthetic cyclodextrines, e.g. β-hydroxypropyl cyclodextrine. A novel coprecipitate of amorphous rosiglitazone maleate with a pharmaceutically acceptable carrier are obtained in the form of white powders after spray-drying processing.

The average molecular weight of polyvinylpyrrolidone (PVP) is not critical and any average molecular weight of PVP (see e.g. The Merck Index, 13^{th} Ed (2001), no. 7783 or Handbook of Pharmaceutical Excipients, 2^{nd} Ed (1994), 392-399, American Pharmaceutical Association Washington and The Pharmaceutical Press London) may be used, but preferably PVP ranges from 10,000 to 100,000 because the capability of preventing crystallization of rosiglitazone maleate and the solubility in the solvent are well balanced.

An appropriate ratio of rosiglitazone maleate to a pharmaceutically acceptable carrier, e.g. PVP, ranges from 1:1 to 1:20 parts by weight, preferably from 1:1 to 1:10, more preferably from 1:1 to 1:4.

A further aspect of the present invention relates to a process for the preparation of said novel coprecipitate of amorphous rosiglitazone maleate with a pharmaceutically acceptable carrier, which comprises the steps of:
a) dissolving rosiglitazone maleate in an organic solvent or in an aqueous solution of organic solvent,
b) adding pharmaceutically acceptable carrier,
c) spray-drying the obtained solution.

During the spray-drying process starting crystalline rosiglitazone maleate transforms to amorphous rosiglitazone maleate and forms a novel coprecipitate of the invention.

Starting crystalline rosiglitazone maleate for said process may be prepared according to the teaching of WO 94/05659. An isomer or tautomeric form and/or a pharmaceutically acceptable solvate of rosiglitazone maleate may be used as starting compound as well.

A still further aspect of the invention relates to a variant process for the preparation of said novel coprecipitate of amorphous rosiglitazone maleate with a pharmaceutically acceptable carrier which comprises the steps of:
a) dissolving rosiglitazone (in the form of base) in an organic solvent or in an aqueous solution of organic solvent
b) adding maleic acid and stirred the mixture to obtain a clear solution,
c) adding pharmaceutically acceptable carrier,
d) spray-drying the obtained solution.

Starting rosiglitazone in the base form may be prepared according to the teaching of EP 306228 A1.

Suitable solvents for use herein include any solvents in which the active compound is soluble. Preferred solvents include any solvents in which the active compound and the carrier are soluble, e.g. ethanol and acetone, preferably used in the range from about 9:1 to 1:1 (V/V), more preferably from about 9:1 to about 7:3 V/V (volume/volume) of organic solvent to water.

During the process of preparing said novel coprecipitate of amorphous rosiglitazone maleate with a pharmaceutically acceptable carrier particles size are reduced what can result in an enhanced dissolution rate due to both increase in the surface area and solubilization. The present invention provides that a crystalline starting rosiglitazone maleate may be stabilized in its amorphous form.

The amorphous form of rosiglitazone maleate in a novel coprecipitate with a pharmaceutically acceptable carrier was detected by X-ray powder diffraction diagrams, measured using a AXS-Bruker D-8 diffractometer (Cu-radiation, Bragg-Brentano Optics, 40 kV, 40 mA, steps 0.01°, time 2 seconds, cut-off: 40°, standard sample carrier).

X-ray powder diffraction analyses were additionally repeated twice for each sample in order to test the stability under ambient conditions and X-ray radiation. No changes in X-ray powder diffraction patterns were observed. Analyses were carried out by means of software DiffracPlus.

All obtained analyses of a novel co-precipitate of amorphous rosiglitazone maleate with polyvinylpyrrolidone, silicon dioxide, mannitol, lactose, methylcellulose or gamma-cyclodextrin show X-ray amorphous pattern. The structures of amorphous patterns are little different what may depend on pharmaceutically acceptable carrier used.

Novel coprecipitate of amorphous rosiglitazone maleate with mannitol show crystalline pattern and one amorphous background. The pattern is not in accord with starting crystalline mannitol and also not in accord with starting crystalline rosiglitazone maleate. The reason for this may be the transformation of mannitol into the mixture of α-D-mannitol and δ (delta)-D-mannitol during the process and the remaining background is a guidance that amorphous rosiglitazone maleate is present.

The present invention produce compositions of a pharmaceutically acceptable carrier in which rosiglitazone maleate is stabilized in its amorphous form. The reduced size of particles and quality of coprecipitate provide for a high surface area of active compound what may result in improved bioavailability.

A further aspect of the present invention are a novel stable pharmaceutical compositions, which may be in the form of suspensions, solutions, elixirs or solid dosage forms, e.g. tablets, capsules, parenteral dosage forms, comprising co-precipitate of amorphous rosiglitazone maleate with a pharmaceutically acceptable carrier and other suitable excipients. Other excipients may be included in the pharmaceutical formulations to further improve the stabilization and/or de-agglomeration of the amorphous particles of active substance. An absorption enhancer as other excipient may be included in the solid dosage forms as well. A preferred oral solid dosage form is a tablet.

Unit dosage of amorphous rosiglitazone maleate in a coprecipitate and/or in a pharmaceutical solid dosage form may range from about 0.1 mg to about 2 g, more preferably from about 2 mg to 8 mg of active compound (rosiglitazone in the form of base).

A still further aspect of the invention relates to a novel coprecipitate of amorphous rosiglitazone maleate with a pharmaceutically acceptable carrier for use in the treatment and/or prophylaxis of diabetes mellitus, conditions associated with diabetes mellitus and certain complications thereof.

A further aspect of the invention relates to a novel stable solid solution of rosiglitazone maleate with a suitable pharmaceutically acceptable carrier. As pharmaceutical acceptable carrier for preparing solid solutions of the invention may be used any materials described in above cited Encyclopedia of Pharmaceutical Technology (Table 1 on page 345), but preferably polyethylene glycols (PEG), PEG from 4000 to 40.000 of average mol wt., more preferably PEG 4000 (see e.g. The Merck Index, 13^{th} Ed (2001), no. 7651). Solid mass of a solid solution of rosiglitazone maleate in an inert carrier are obtained.

A process for the preparation of a solid solution comprising the steps of:
a) melting rosiglitazone maleate and a pharmaceutically acceptable carrier to form a melt
b) cooling the obtained melted solution
   or optionally the process variant, which comprises the steps of:
a) melting rosiglitazone (base), maleic acid and a pharmaceutically acceptable carrier to form a melt
b) cooling the obtained melted solution

Said novel solid solution of the invention can be used for the preparation of pharmaceutical compositions, e.g. solid dosage forms, preferably tablets, which further comprises other suitable excipients, for use in the treatment and/or prophylaxis of diabetes mellitus, conditions associated with diabetes mellitus and certain complications thereof.

We have surprisingly and unexpectedly found that an exacting process of preparing a pharmaceutical compositions comprising an electrospun fiber of a pharmaceutically acceptable polymeric carrier homogeneously integrated with a stable amorphous form of an active agent as described in WO 04/014304 may be avoided. Instead of electrospinning method much simpler method of spray-drying is used and also an other suitable pharmaceutically carrier may be used besides polymers.

The following examples illustrate the invention but do not limit it in any way.

### EXAMPLE 1

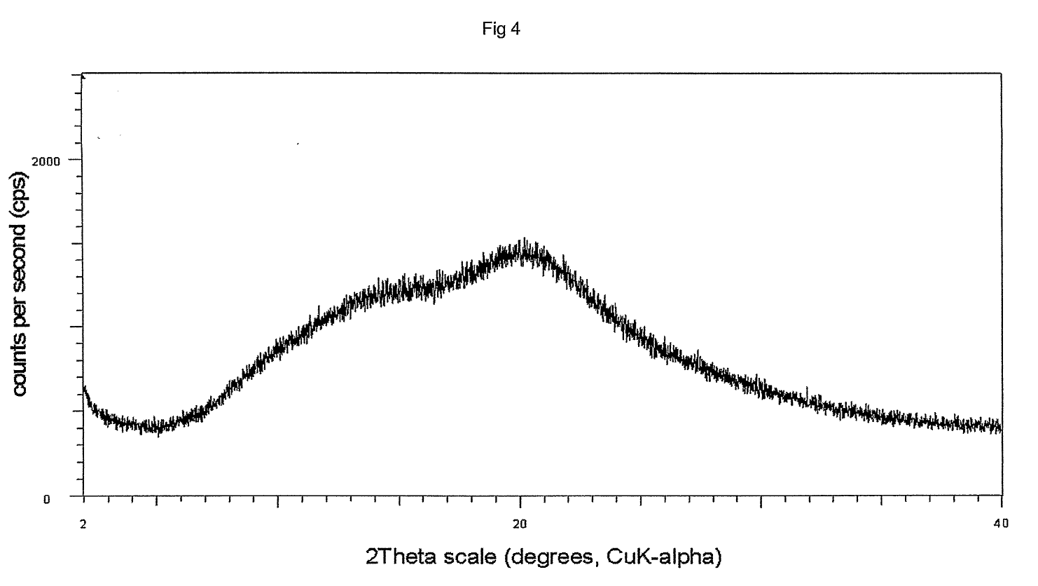

Rosiglitazone maleate (5.0 g) is dissolved in 150 ml of ethanol (96%) and water (9:1, V/V) and stirred until a clear solution is obtained. To the obtained solution Polyvidone K-30 [polyvinylpyrrolidone] (10.0 g) is added and stirred again until a solution is obtained. The obtained solution is spray-dried on a mini spray-dryer (Büchi 190). White powder of coprecipitate of amorphous rosiglitazone maleate with polyvinylpyrrolidone presented as X-ray powder diffraction pattern on FIG. 4 is obtained.

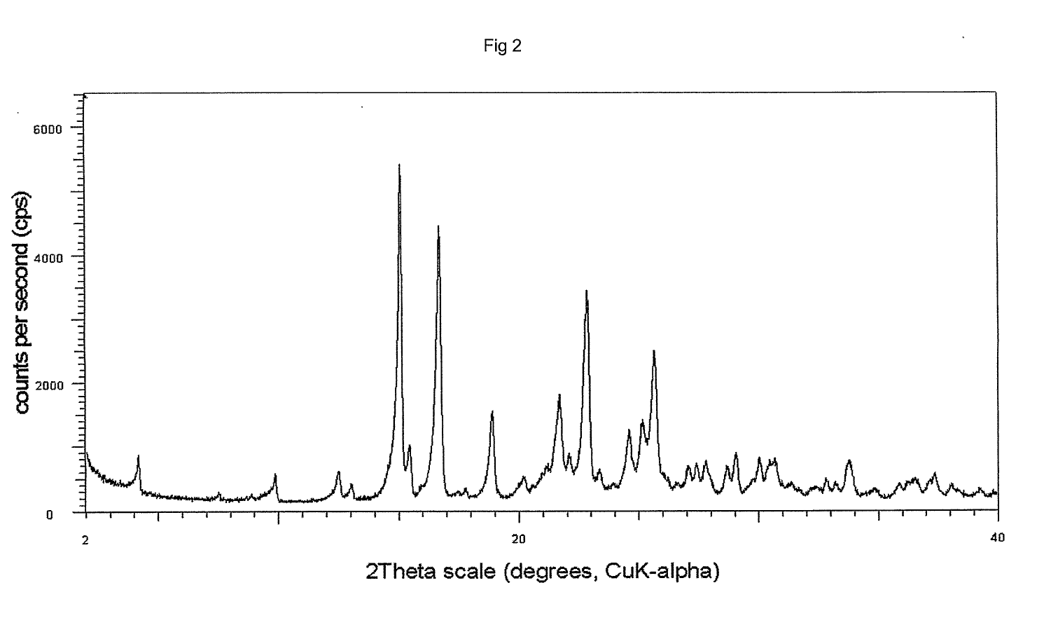

Starting rosiglitazone maleate is crystalline compound presented as X-ray diffraction pattern on FIG. 2.

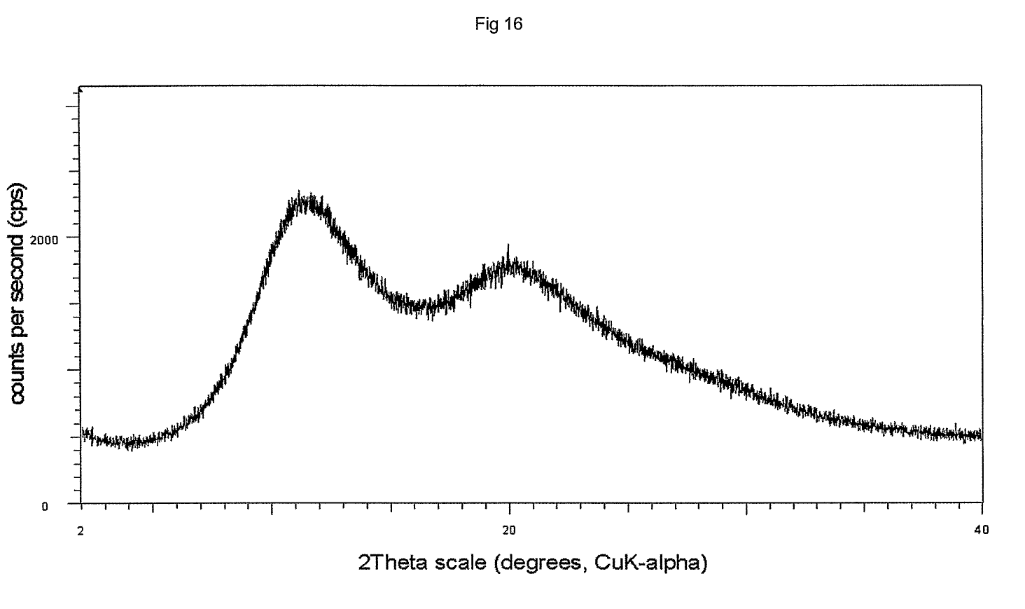

Polyvidon K30 is amorphous compound presented as X-ray powder diffraction pattern on FIG. 16.

### EXAMPLE 2

Rosiglitazone maleate (5.0 g) is dissolved in 150 ml acetone and water (7:3, V/V) and stirred until a clear solution is obtained. To the obtained solution Polyvidone K-30 [polyvinylpyrrolidone] (10.0 g) is added and stirred again until a solution is obtained. The obtained solution is spray-dried on a mini spray-dryer (Büchi 190). White powder of coprecipitate of amorphous rosiglitazone maleate with polyvinylpyrrolidone is obtained.

### EXAMPLE 3

Rosiglitazone maleate (5.0 g) is dissolved in 300 ml of ethanol (96%) and water (9:1, V/V) and stirred until a clear solution is obtained. To the obtained solution Polyvidon K-30 [polyvinylpyrrolidone] (20.0 g) is added and stirred again until a solution is obtained. The obtained solution is spray-dried on a mini spray-dryer (Büchi). White powder of coprecipitate of amorphous rosiglitazone maleate with polyvinylpyrrolidone is obtained.

### EXAMPLE 4

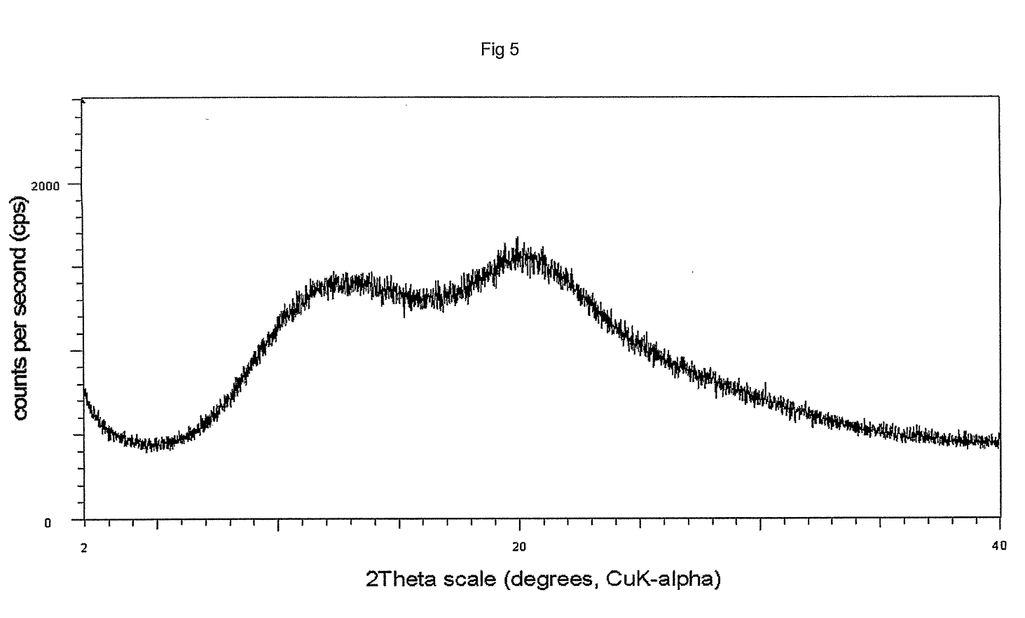

Rosiglitazone maleate (5.0 g) is dissolved in 300 ml of acetone and water (7:3, V/V) and stirred until a clear solution is obtained. To the obtained solution Polyvidone K-30 [polyvinylpyrrolidone] (20.0 g) is added and stirred again until a solution is obtained. The solution is spray-dried on a mini spray-dryer (Büchi). White powder of co-precipitate of amorphous rosiglitazone maleate with polyvinylpyrrolidone presented on FIG. 5 is obtained.

### EXAMPLE 5

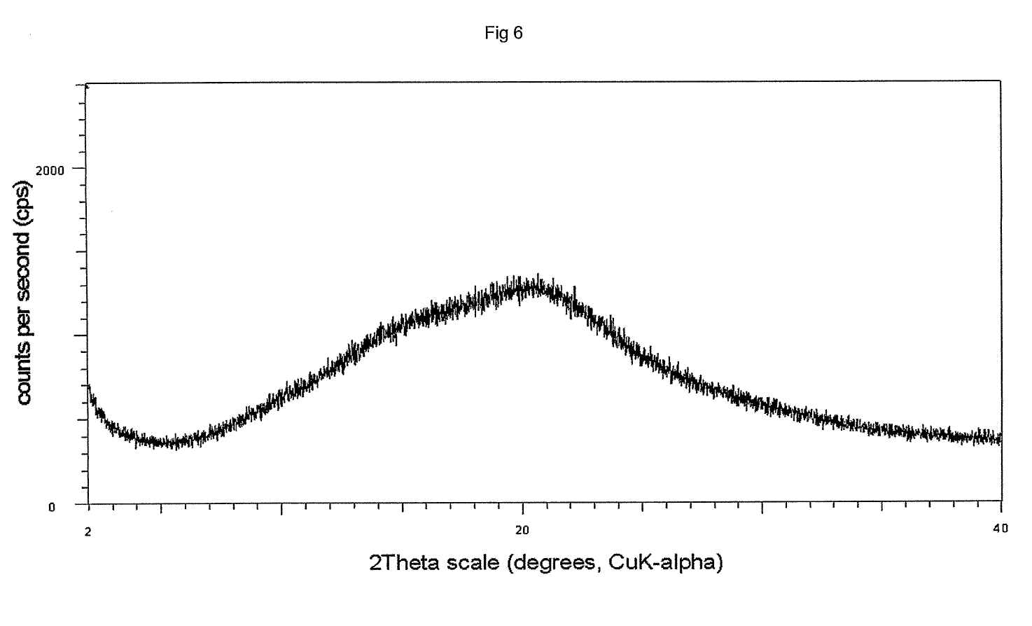

Rosiglitazone maleate (5.0 g) is dissolved in 150 ml of ethanol (96%) and water (9:1, V/V) and stirred until a clear solution is obtained. To the obtained solution Polyvidone K-30 [polyvinylpyrrolidone] (5.0 g) is added and stirred again until a solution is obtained. The obtained solution is spray-dried on a mini spray-dryer (Büchi). White powder of coprecipitate of amorphous rosiglitazone maleate with polyvinylpyrrolidone presented on FIG. 6 is obtained.

### EXAMPLE 6

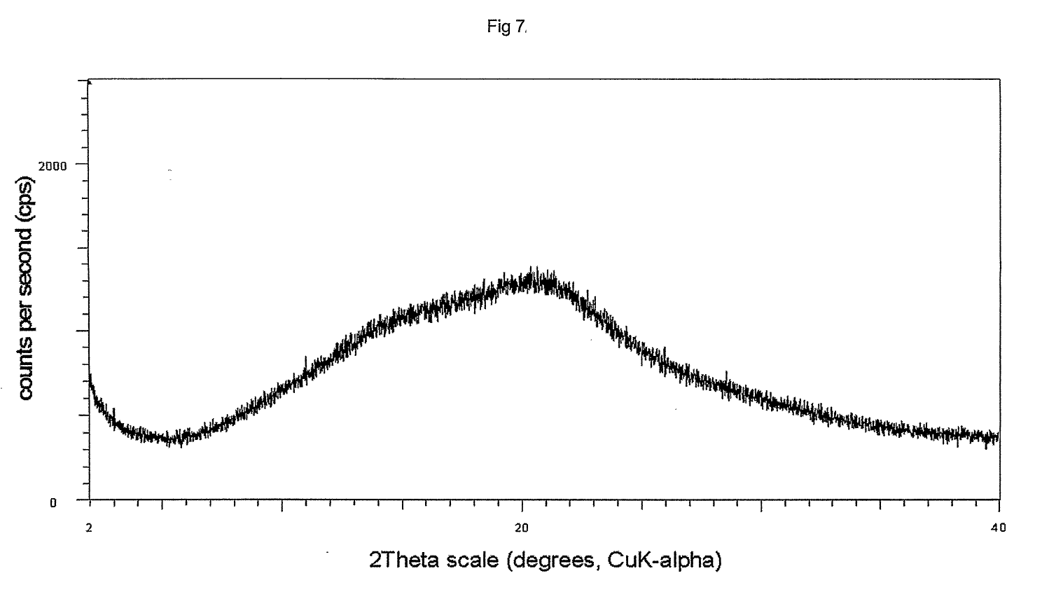

Rosiglitazone maleate (5.0 g) is dissolved in 150 ml of acetone and water (7:3, V/V) and stirred until a clear solution is obtained. To the obtained solution Polyvidone K-30 [polyvinylpyrrolidone] (5.0 g) is added and stirred again until a solution is obtained. The obtained solution is spray-dried on a mini spray-dryer (Büchi). White powder of coprecipitate of amorphous rosiglitazone maleate with polyvinylpyrrolidone presented on FIG. 7 is obtained.

### EXAMPLE 7

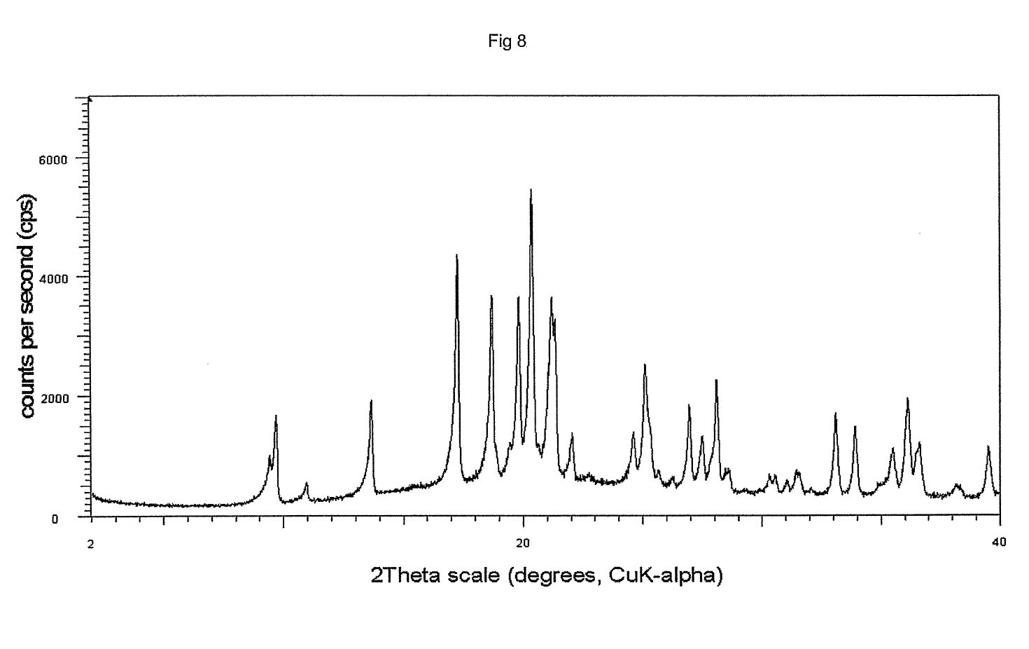

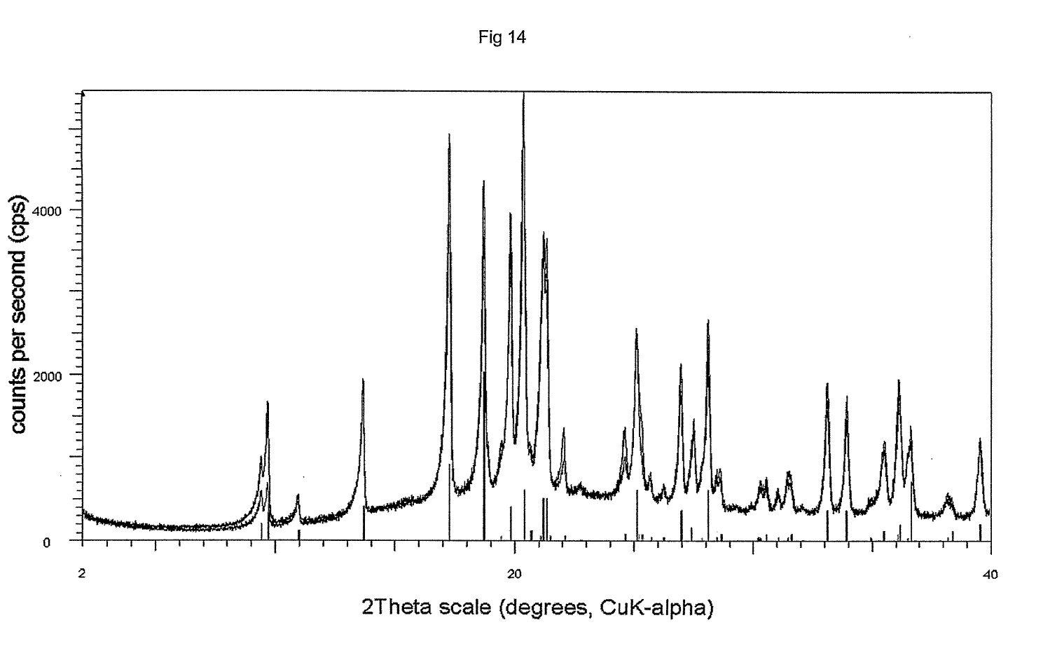

Rosiglitazone maleate (5.0 g) is dissolved in 300 ml of ethanol (96%) and water (1:1, V/V) and stirred until a clear solution is obtained. To the obtained solution Mannit [mannitol] (10.0 g) is added and stirred again until a solution is obtained. The obtained solution is spray-dried on a mini spray-dryer (Büchi). White powder of co-precipitate of amorphous rosiglitazone maleate with mannitol presented as X-ray powder diffraction pattern on FIG. 8 is obtained. X-ray diffraction pattern on FIG. 14 shows that in the remaining background amorphous rosiglitazone maleate is present.

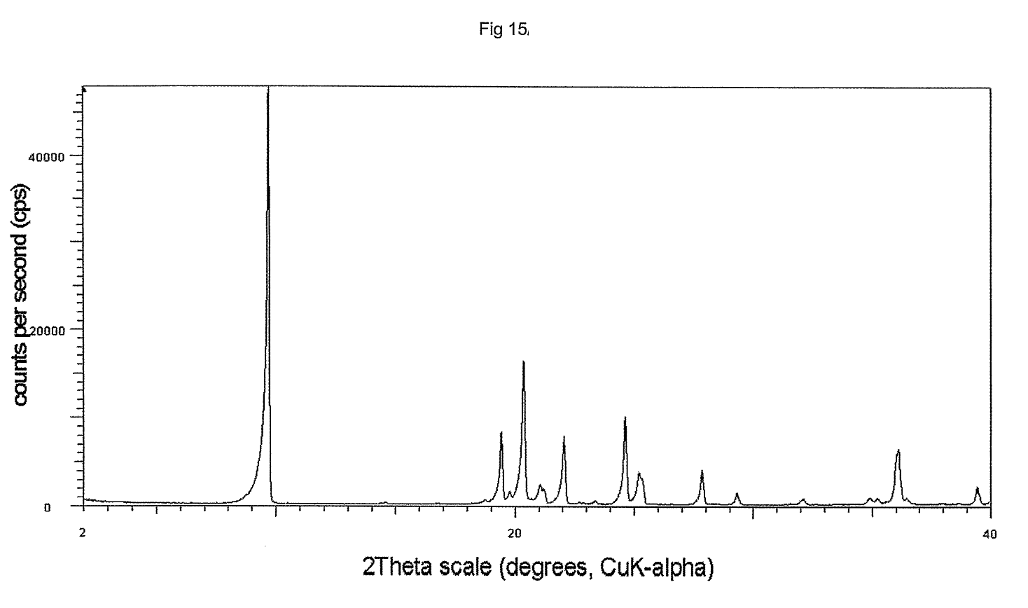

Mannit (Merck) is crystalline compound presented as X-ray diffraction pattern on FIG. 15.

### EXAMPLE 8

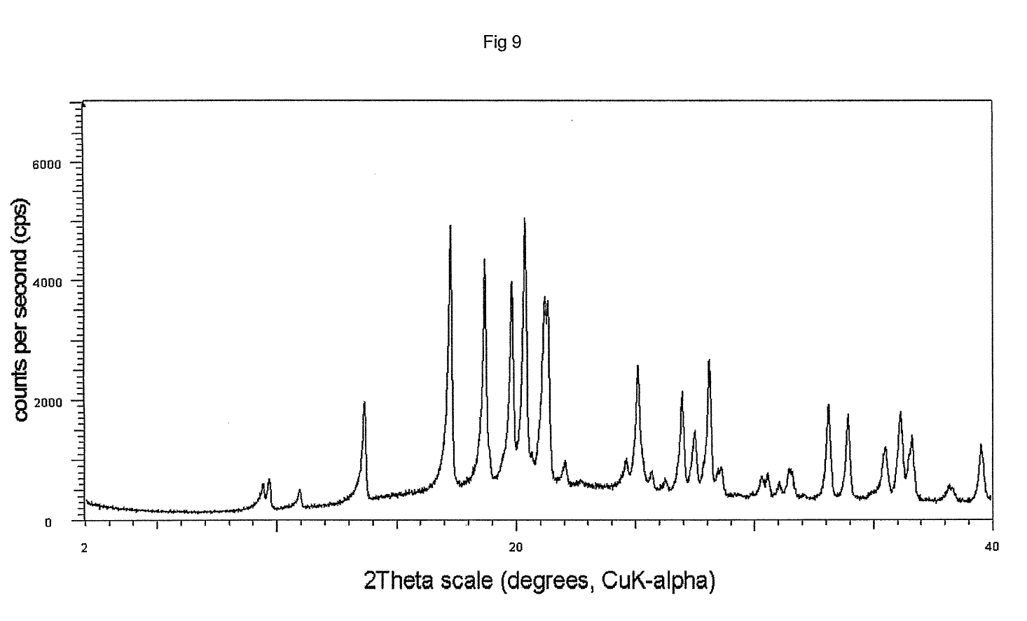

Rosiglitazone maleate (5.0 g) is dissolved in 300 ml of acetone and water (1:1, V/V) and stirred until a clear solution is obtained. To the obtained solution Mannit [mannitol] (10.0 g) is added and stirred again until a turbid solution is obtained. The obtained turbid solution is spray-dried on a mini spray-dryer (Büchi). White powder of coprecipitate of amorphous rosiglitazone maleate with mannitol presented as X-ray powder diffraction pattern on FIG. 9 is obtained.

### EXAMPLE 9

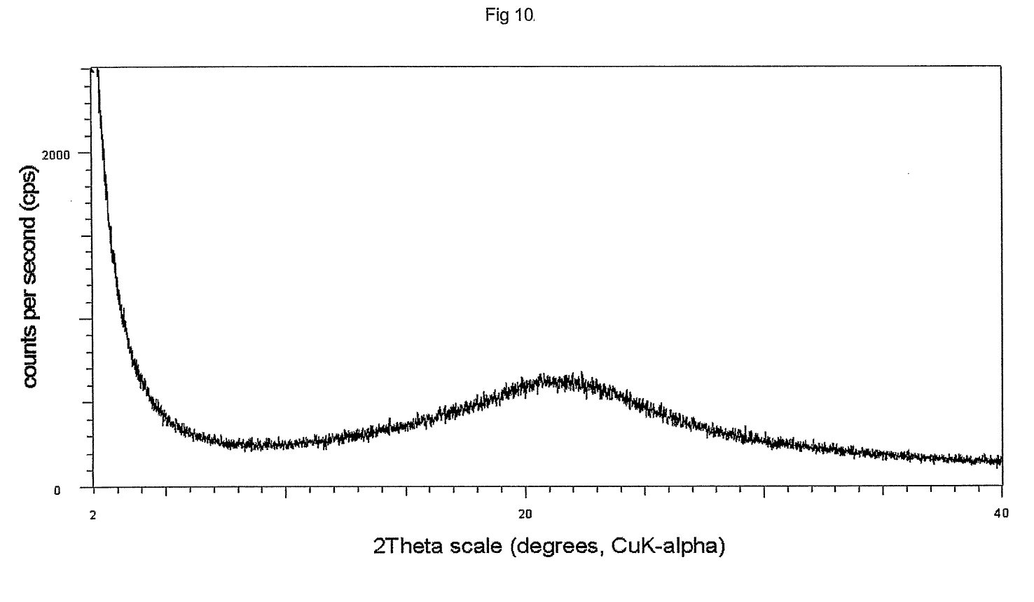

Rosiglitazone maleate (5.0 g) is dissolved in 300 ml of acetone and water (9:1, V/V) and stirred until a clear solution is obtained. To the obtained solution Aerosil 200 [silicon dioxide] (10.0 g) is added and stirred for 10 minutes. The obtained suspension is spray-dried with stirring on a mini spray-dryer (Büchi 190). White powder of coprecipitate of amorphous rosiglitazone maleate with silicon dioxide presented as X-ray powder diffraction pattern on FIG. 10 is obtained.

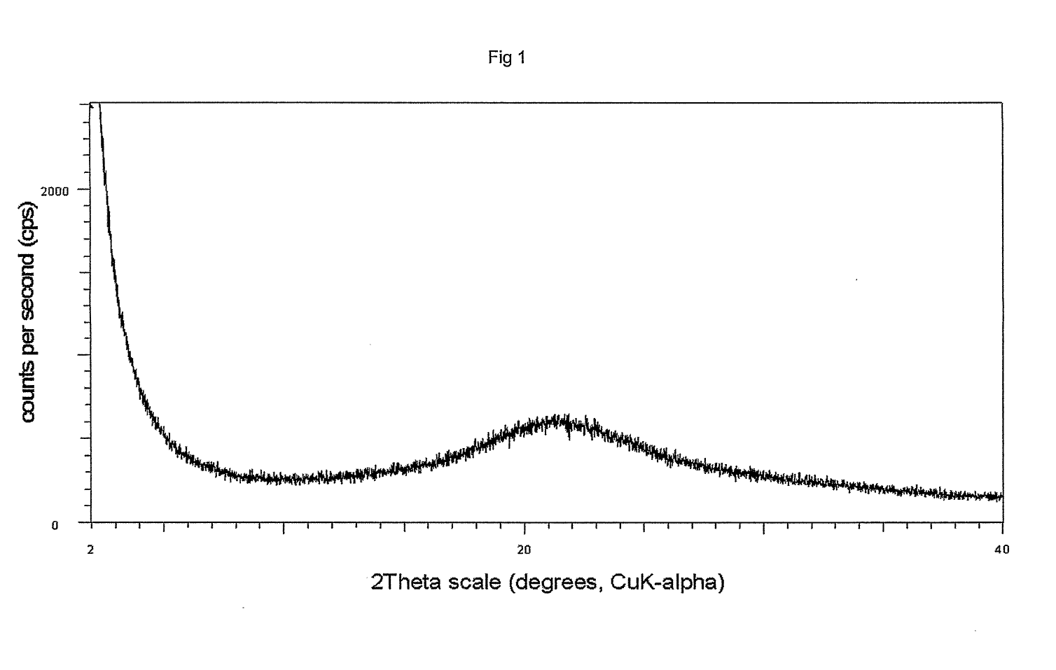

Aerosil 200 is amorphous compound presented as X-ray powder diffraction pattern on FIG. 1.

### EXAMPLE 10

Rosiglitazone maleate (5.0 g) is dissolved in 300 ml of acetone and water (9:1, V/V) and stirred until a clear solution is obtained. To the obtained solution Aerosil 200 [silicon dioxide] (5.0 g) is added and stirred for 10 minutes. The obtained suspension is spray-dried with stirring on a mini spray-dryer (Büchi). White powder of coprecipitate of amorphous rosiglitazone maleate with silicon dioxide is obtained.

### EXAMPLE 11

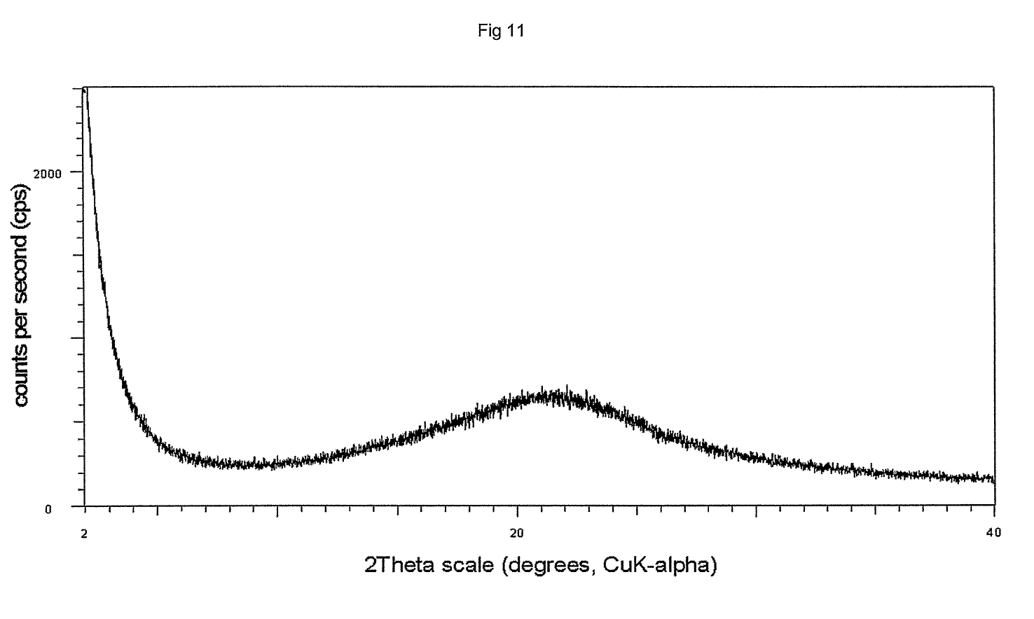

Rosiglitazone maleate (5.0 g) is dissolved in 300 ml of ethanol (96%) and water (9:1, V/V) and stirred until a clear solution is obtained. To the obtained solution Aerosil 200 [silicon dioxide] (5.0 g) is added and stirred for 10 minutes. The obtained suspension is spray-dried with stirring on a mini spray-dryer (Büchi). White powder of coprecipitate of amorphous rosiglitazone maleate with silicon dioxide presented as X-ray diffraction pattern on FIG. 11 is obtained.

### EXAMPLE 12

Rosiglitazone maleate (5.0 g) is dissolved in 300 ml of ethanol (96%) and water (9:1, V/V) and stirred until a clear solution is obtained. To the obtained solution Aerosil 200 [silicon dioxide] (10.0 g) is added and stirred for 10 minutes. The obtained suspension is spray-dried with stirring on a mini spray-dryer (Büchi). White powder of coprecipitate of amorphous rosiglitazone maleate with silicon dioxide is obtained.

### EXAMPLE 13

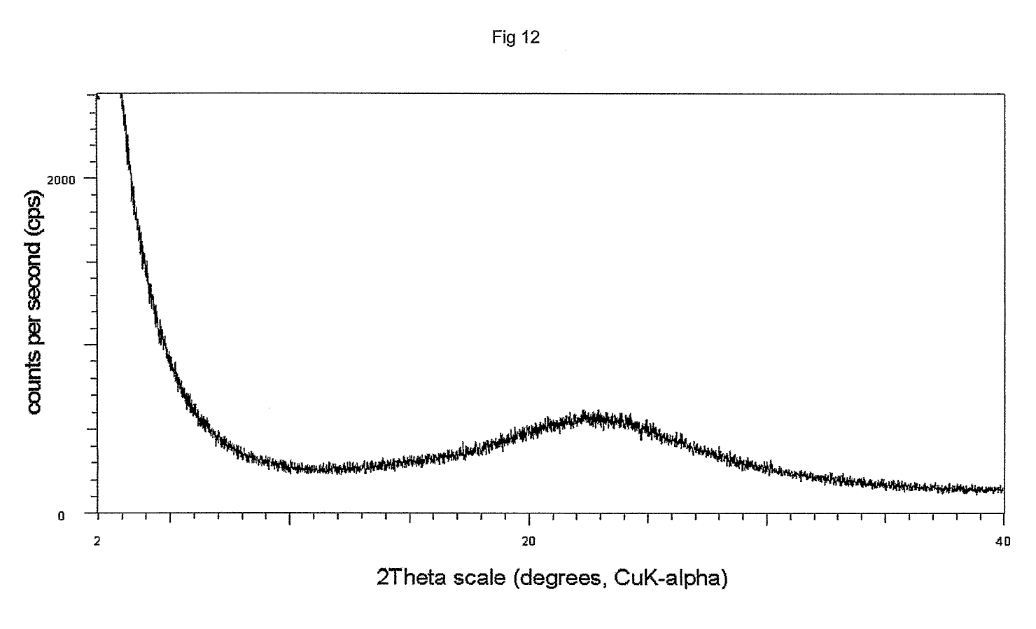

Rosiglitazone maleate (5.0 g) is dissolved in 300 ml of acetone and water (9:1, V/V) and stirred until a clear solution is obtained. To the obtained solution Syloid [amorphous silicon dioxide] (20.0 g) is added for 10 minutes. The obtained suspension is spray-dried with stirring on a mini spray-dryer (Büchi). White powder of coprecipitate of amorphous rosiglitazone maleate with silicon dioxide presented as X-ray powder diffraction pattern on FIG. 12 is obtained.

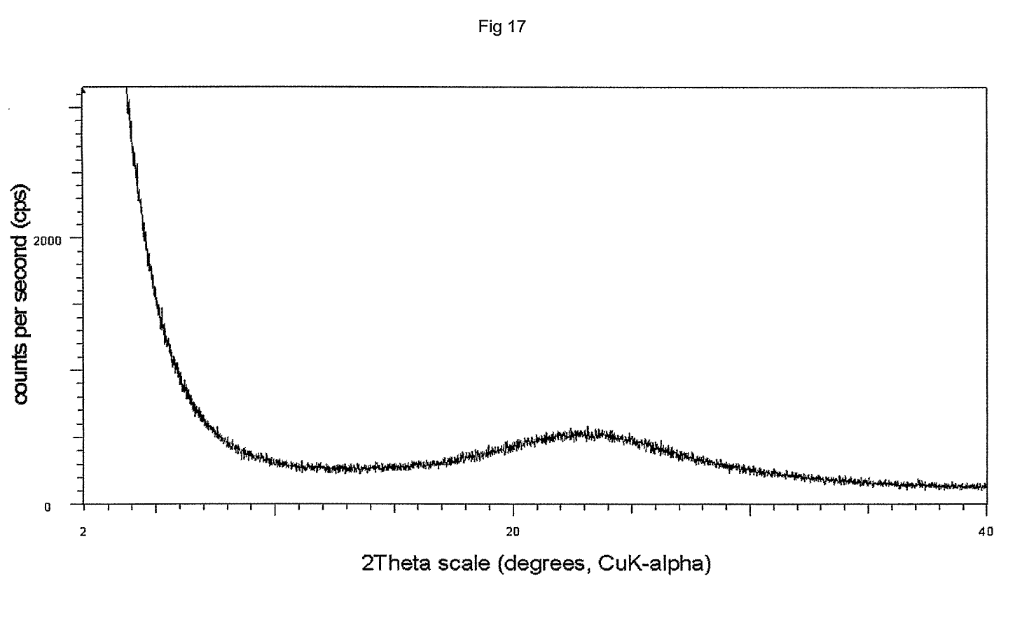

Syloid is amorphous compound presented as X-ray powder diffraction pattern on FIG. 17.

### EXAMPLE 14

Rosiglitazone maleate (5.0 g) is dissolved in 300 ml of ethanol (96%) and water (9:1, V/V) and stirred until a clear solution is obtained. To the obtained solution Syloid [amorphous silicon dioxide] (20.0 g) is added and stirred for 10 minutes. The obtained suspension is spray-dried with stirring on a mini spray-dryer (Büchi). White powder of coprecipitate of amorphous rosiglitazone maleate with silicon dioxide is obtained.

### EXAMPLE 15

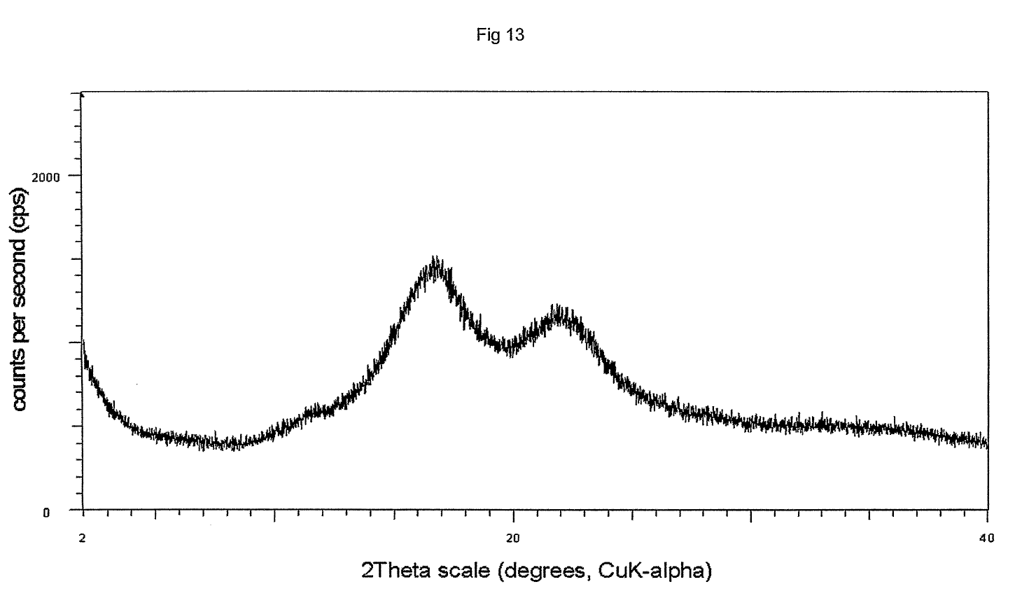

Rosiglitazone maleate (5.0 g) is dissolved in 150 ml of ethanol (96%) and 50 ml water and stirred until a clear solution is obtained. To the obtained solution a solution of Cavamax W8 [gamma-Cyclodextrin] (10.0 g) in 100 ml water is added and stirred again for 10 minutes. The obtained solution is spray-dried on a mini spray-drier (Büchi). White powder of coprecipitate of amorphous rosiglitazone maleate with gamma cyclodextrin as presented on FIG. 13 is obtained.

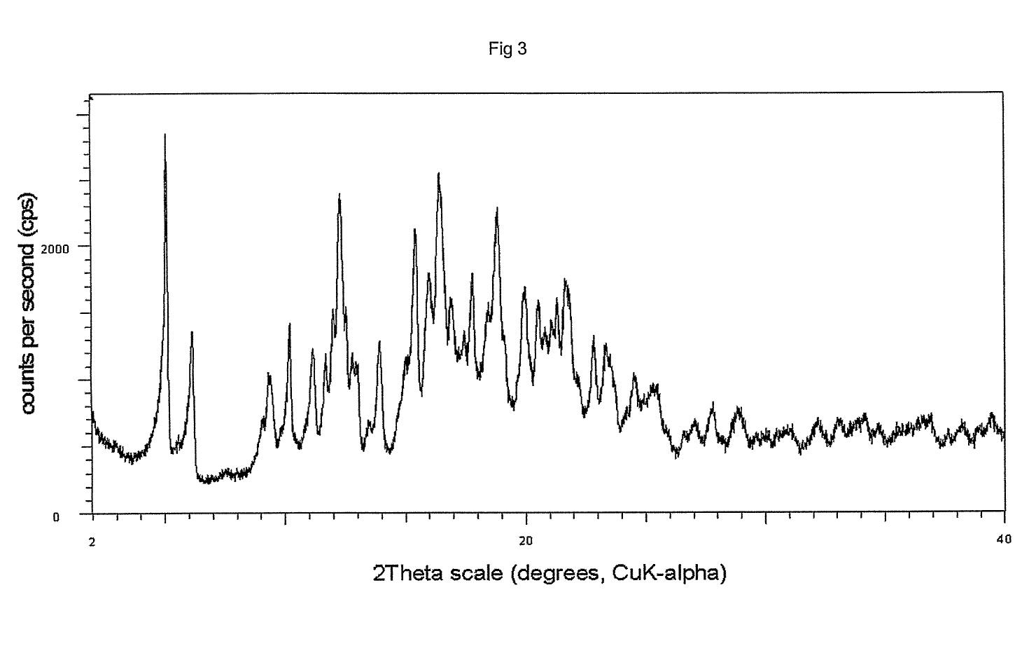

Cavamax W8 is crystalline compound presented as X-ray powder diffraction pattern on FIG. 3.

### EXAMPLE 16

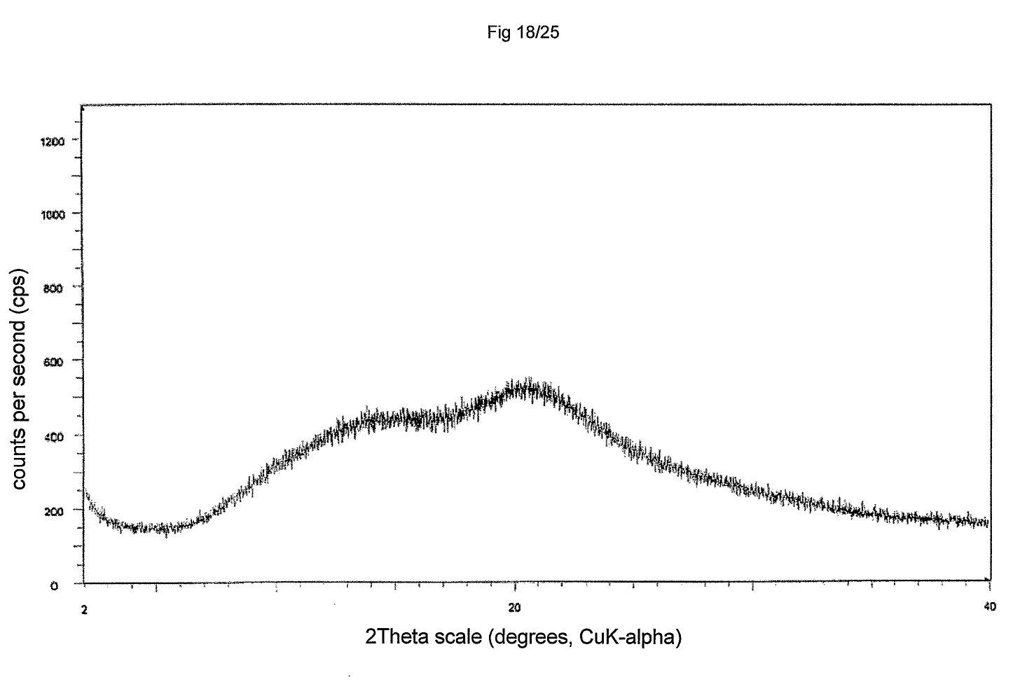

Rosiglitazone in base form (3.77 g) is dissolved in 250 ml of ethanol and water (9:1, V/V), maleic acid (1.22 g) is added and the mixture is stirred until a clear solution is obtained. To the obtained solution Polyvidone K-30 [polyvinylpirrolidone] (20.0 g) is added and stirred again until a solution is obtained. The obtained solution is spray-dried on a mini spray-dryer (Büchi 190). White powder of coprecipitate of amorphous rosiglitazone maleate with polyvinylpyrrolidone (7.7 g) presented on FIG. 18 is obtained.

### EXAMPLE 17

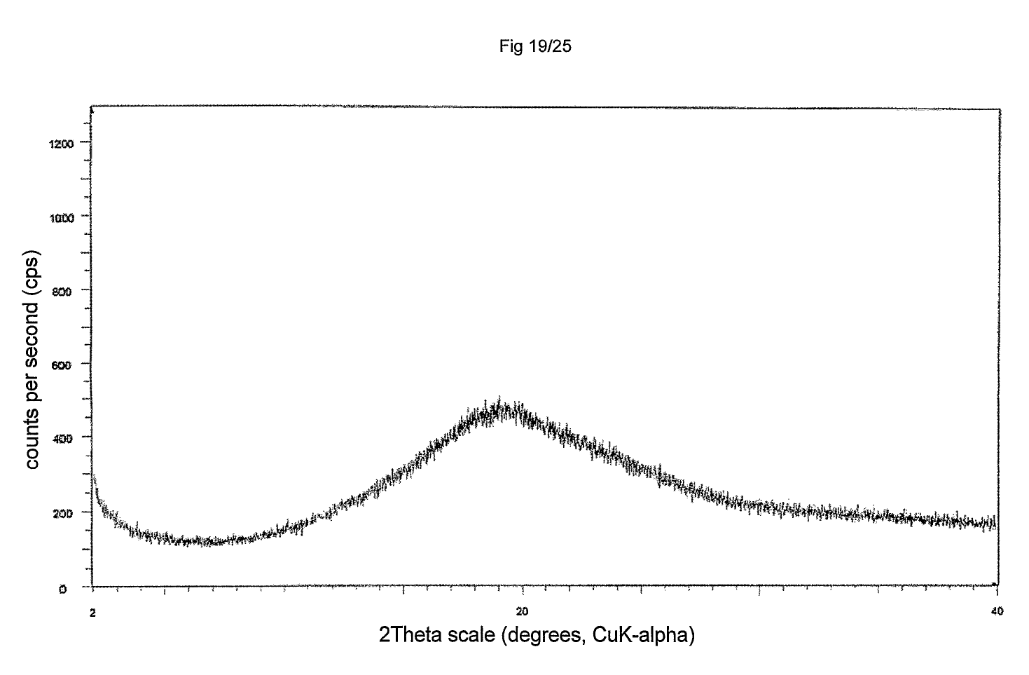

Rosiglitazone maleate (5.0 g) is dissolved in 150 ml of ethanol and 50 ml of water and stirred until a clear solution is obtained. To the obtained solution a solution of lactose (10.0 g) in 150 ml water is added and stirred again for 10 min. The obtained solution is spray-dried on a mini spray-dryer (Büchi) and an obtained powder is further dried in vacuum at ambient temperature over night. White powder of co-precipitate of amorphous rosiglitazone maleate with methylcellulose presented on FIG. 19 is obtained.

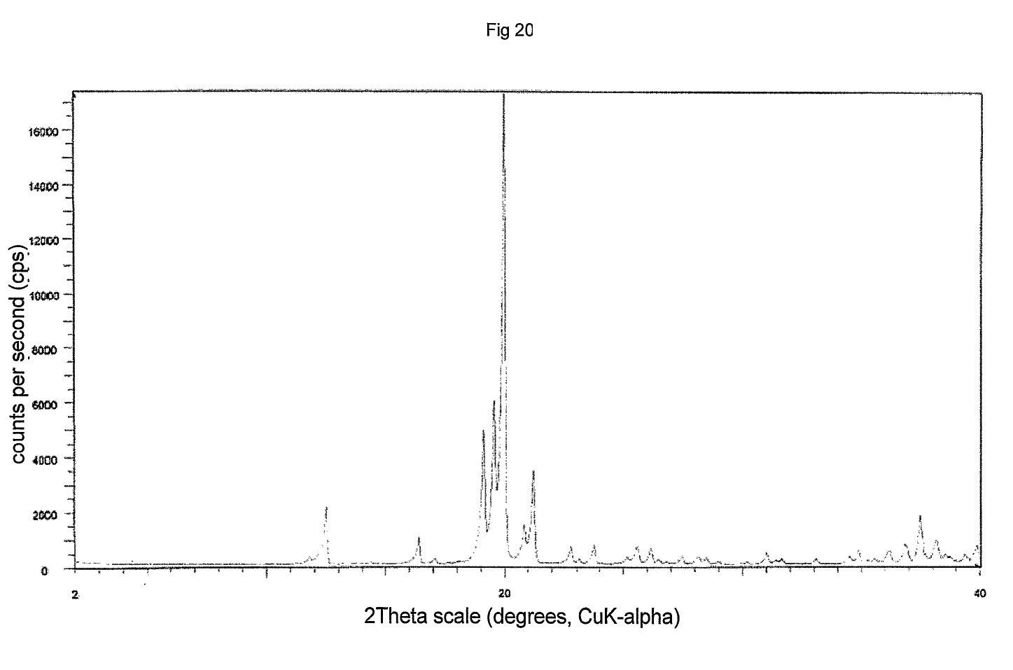

Lactose (lactose hydrate) is crystalline compound presented as X-ray diffraction pattern on FIG. 20.

### EXAMPLE 18

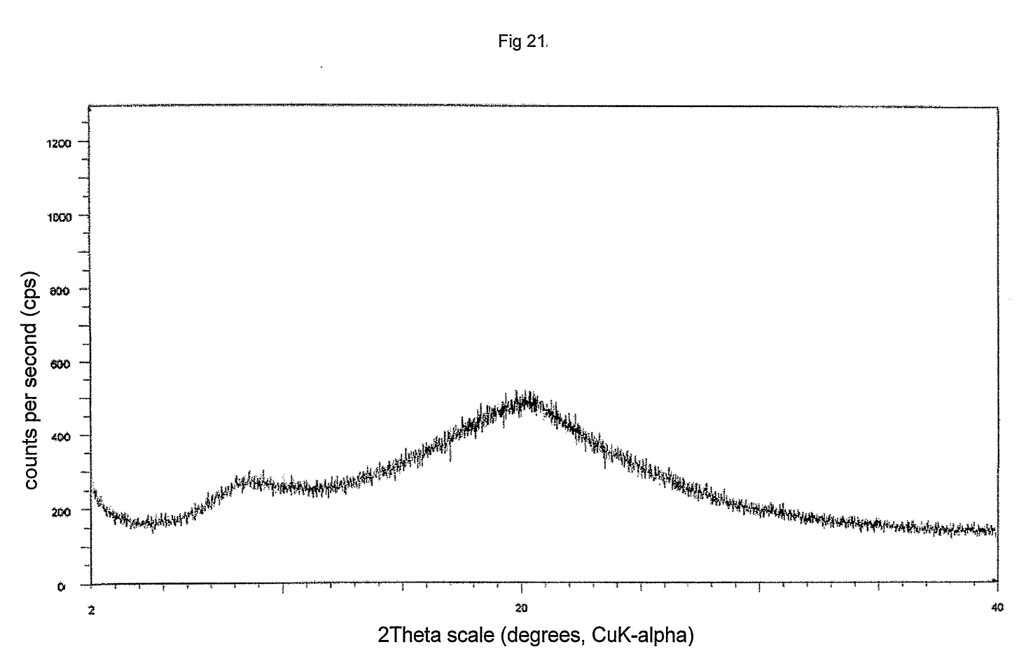

Rosiglitazone maleate (5.0 g) is dissolved in 450 ml of ethanol and 50 ml of water and stirred until a clear solution is obtained. To the obtained solution methylcellulose (10.0 g) is added and stirred again for 10 min. The obtained suspension is spray-dried on a mini spray-dryer (Büchi 190). White powder of coprecipitate of amorphous rosiglitazone maleate with lactose presented on FIG. 21 is obtained.

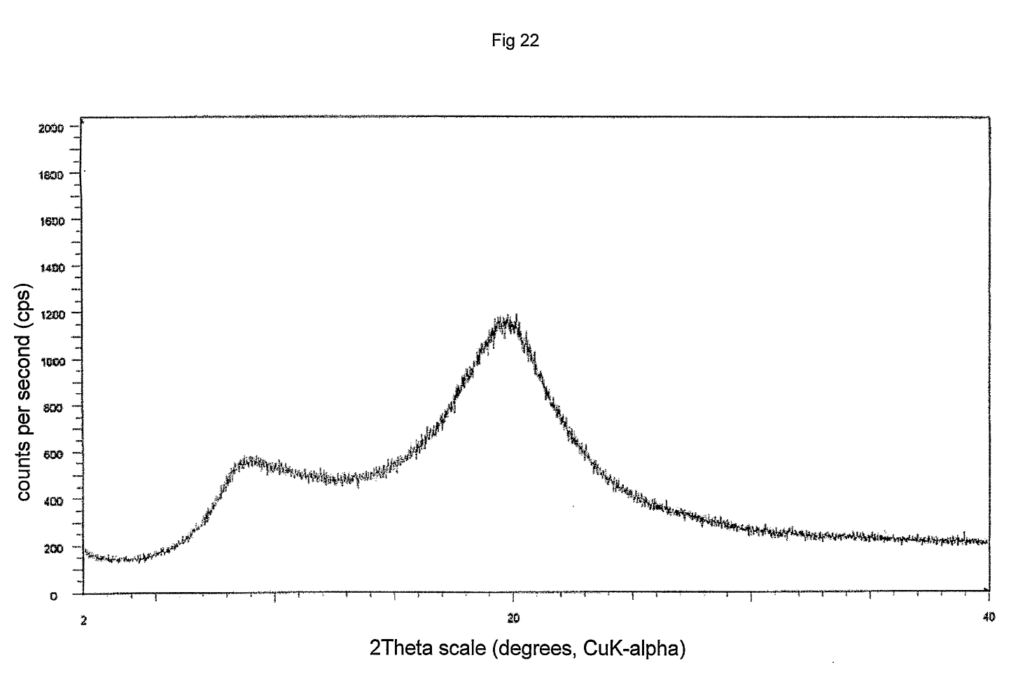

Methylcellulose is amorphous compound presented as X-ray diffraction pattern on FIG. 22.

### EXAMPLE 19

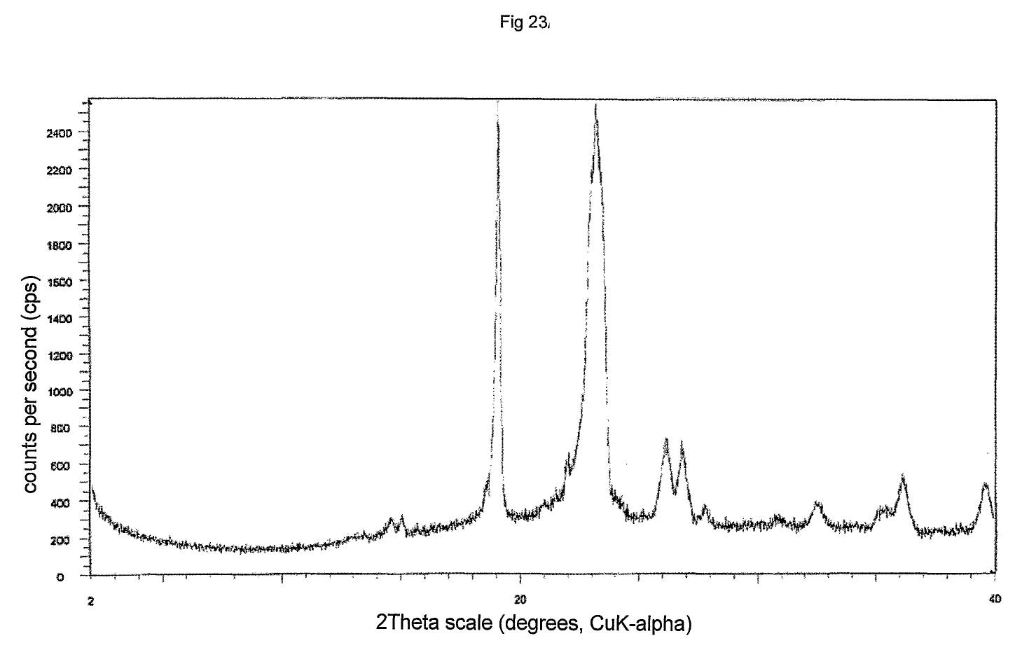

10 g of PEG 400 [polyethylene glycol 400], 0.75 g of rosiglitazone (in base form) and 0.25 g of maleic acid are mixed and the resulted mixture is heated to 80° C. The obtained solution is then allowed to cool to room temperature and solid mass of solid solution of rosiglitazone maleate presented on FIG. 23 is obtained.

### EXAMPLE 20

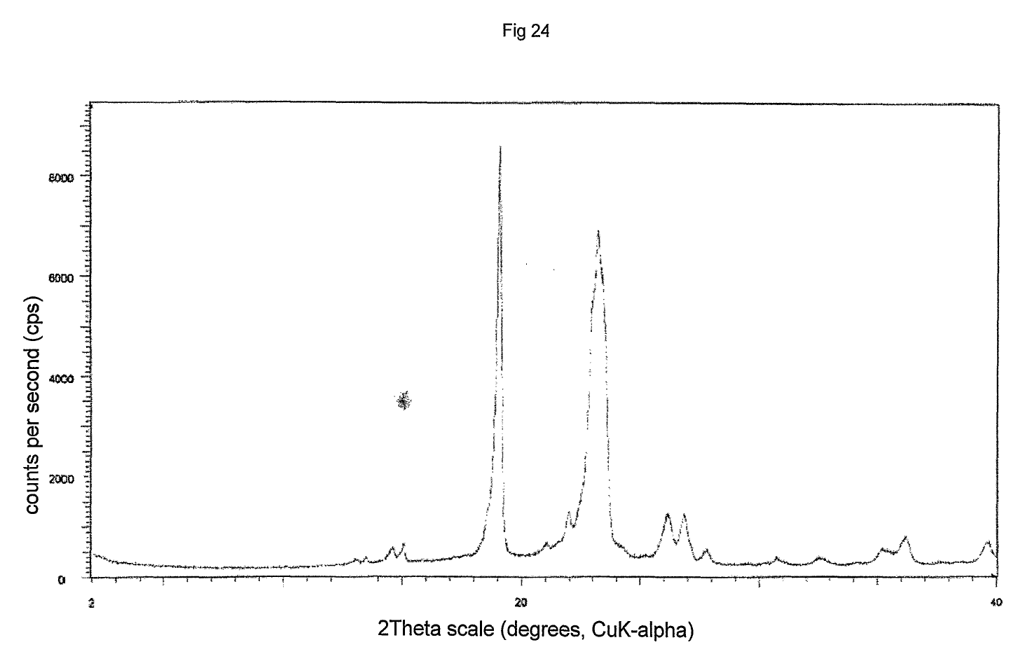

18 g of PEG 4000 and 0.5 g of rosiglitazone maleate are heated to 80° C. The obtained solution is then allowed to cool to room temperature and solid mass of solid solution of rosiglitazone maleate presented on FIG. 24 is obtained.

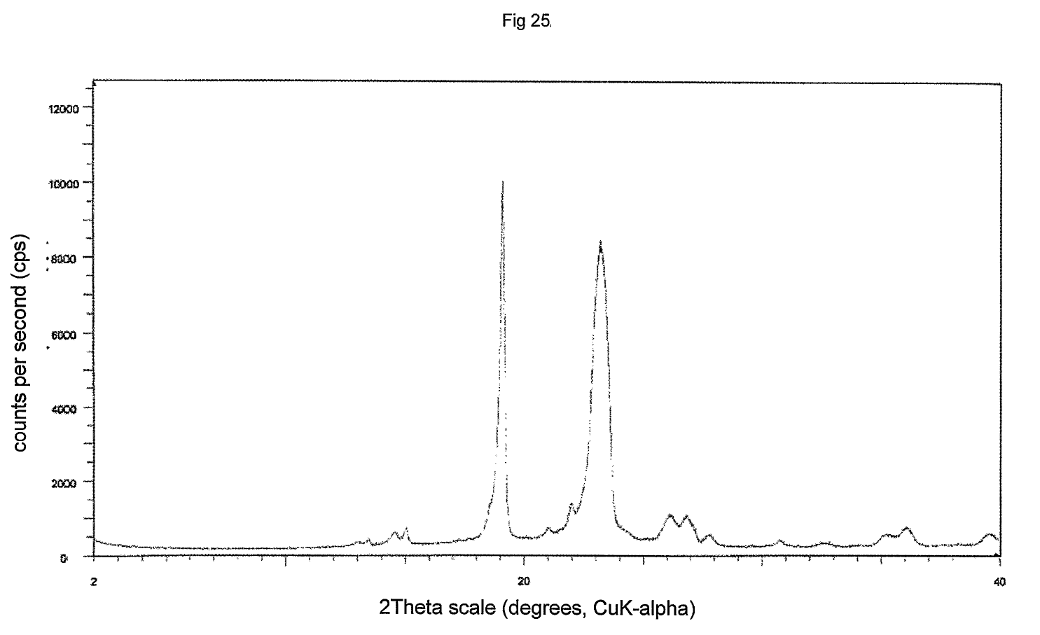

X-ray powder diffraction pattern of PEG 4000 is presented on FIG. 25.

1. A coprecipitate of amorphous rosiglitazone maleate with a pharmaceutically acceptable carrier.

2. A coprecipitate of amorphous rosiglitazone maleate with a pharmaceutically acceptable carrier according to claim 1, wherein the carrier is selected from the group consisting of polyvinylpyrrolidone, silicium dioxide, mannitol, lactose, methylcellulose and cyclodextrin.

3. The coprecipitate according to claim 1, wherein it is the coprecipitate of amorphous rosiglitazone maleate with polyvinylpyrrolidone.

4. The coprecipitate according to claim 1, wherein it is the coprecipitate of amorphous rosiglitazone maleate with silicon dioxide.

5. The coprecipitate according to claim 1, wherein it is the coprecipitate of amorphous rosiglitazone maleate with mannitol.

6. The coprecipitate according to claim 1, wherein it is the coprecipitate of amorphous rosiglitazone maleate with lactose.

7. The coprecipitate according to claim 1, wherein it is the coprecipitate of amorphous rosiglitazone maleate with methylcellulose.

8. The coprecipitate according to claim 1, wherein it is the coprecipitate of amorphous rosiglitazone maleate with gamma-cyclodextrin.

9. A coprecipitate according to claim 1, wherein the ratio of amorphous rosiglitazone maleate to a pharmaceutically acceptable carrier ranges from 1:1 to 1:20.

10. A coprecipitate according to claim 1, wherein the ratio of amorphous rosiglitazone maleate to a pharmaceutically acceptable carrier ranges from 1:1 to 1:4.

11. A process for the preparation of a coprecipitate of amorphous rosiglitazone maleate with a pharmaceutically acceptable carrier, which comprises the steps of:
a) dissolving rosiglitazone maleate in an organic solvent or in an aqueous solution of organic solvent,
b) adding pharmaceutically acceptable carrier,
c) spray-drying the obtained solution.

12. The process according to claim 11, wherein a pharmaceutically acceptable carrier is selected from the group consisting of polyvinylpyrrolidone, silicon dioxide, mannitol, lactose, methylcellulose and cyclodextrin.

13. The process according to claim 11, wherein an organic solvent is selected from the group consisting of ethanol and acetone.

14. The process according to claim 11, wherein the range of organic solvent to water is from about 9:1 to about 1:1 (V/V).

15. The process according to claims 11, wherein the range of organic solvent to water is from about 9:1 to about 7:3 (V/V)

16. A process for the preparation of a coprecipitate of amorphous rosiglitazone maleate with a pharmaceutically acceptable carrier, which comprises the steps of:
d) dissolving rosiglitazone (base) in an organic solvent
e) adding maleic acid and stirred the mixture to obtain a clear solution,
f) adding pharmaceutically acceptable carrier,
g) spray-drying the obtained solution.

17. (canceled)

18. A coprecipitate of amorphous rosiglitazone maleate with a pharmaceutically acceptable carrier according to claim 1, for use in the treatment and/or prophylaxis of diabetes mellitus, conditions associated with diabetes mellitus and certain complications thereof.

19. (canceled)

20. A solid solution of rosiglitazone maleate with a pharmaceutically acceptable carrier.

21. A solid solution according to claim 20, wherein the pharmaceutically acceptable carrier is selected from polyethylene glycols between 4000 to 40,000 of average mol. weight.

22. A process for the preparation of a solid solution of rosiglitazone maleate with a pharmaceutical acceptable carrier, which comprises the steps of:
h) melting rosiglitazone maleate or optionally rosiglitazone and maleic acid with a pharmaceutically acceptable carrier to form a melt
i) cooling the obtained melted solution

23-24. (canceled)
